(19) United States
(12) Patent Application Publication
(10) Pub. No.: US 2009/0113596 A1
(43) Pub. Date: May 7, 2009

(54) Compression garment or method of manufacture

(75) Inventor: Greg Young, Bundall (AU)

Correspondence Address: Oppedahl Patent Law Firm LLC, P.O. BOX 4850, FRISCO, CO, 80443-4850, US

(73) Assignee: JEST TECHNOLOGIES PTY LTD, Bundall (AU)

(21) Appl. No.: 12/093,443

(22) PCT Filed: Oct. 30, 2006
(86) PCT No.: PCT/AU2006/001606

§ 371 Date: Oct. 14, 2008

(30) Foreign Application Priority Data
Nov. 25, 2005 (AU) 2005906582

(51) Int. Cl. A41D 1/00; A61F 13/00; D05B 23/00

(52) U.S. Cl. 2/69; 602/61; 112/475.09

(57) ABSTRACT

A compression garment 10 comprising a compression fabric formed into a number of panels 11 to 31, said panels defining muscle groups according to the surface anatomy of a person; and a number of seams 40 joining adjacent panels; such that the shape of the compression garment is formed by joining adjacent said number of panels together at said number of seams; wherein each garment panel provides targeted compressive support to the said muscle groups isolating each muscle group from the others and the seams 40 follow the contour of the surface anatomy of the muscle groups when the garment is assembled and worn.

### TECHNICAL FIELD

The present invention relates to a compression garment or method of manufacture thereof and in particular a compression garment for increasing circulation, or reducing or assisting recovery from soft tissue injury.

### BACKGROUND ART

Sports are an important part of a healthy lifestyle. Playing with a social team encourages camaraderie, and provides purpose, stress relief and exercise in everyday life. Additionally, individual recreational sports provide a goal to strive for to improve individual performance. At the elite level, sports can be a career choice for the best competitors, who treat their sport as a form of employment.

Injury from sports including strains and physiological stresses, can have a dramatic effect, both for social and elite competitors alike. Such injury to the legs muscles such as quadriceps, hamstrings, groin or calves can affect the ability to walk or run, with major injuries to tendons, ligaments and cartilage resulting in the need for surgery. These leg injuries can seriously affect the mobility of a person, whether in performance for competitive sports or in every day life. Further, injuries to shoulder or arm muscles such as the deltoids, triceps and biceps can result in a person having a reduced range in arm movements and in extreme cases requiring reconstructive surgery.

With soft tissue injury, the muscles, cartilage, tendons or ligaments are damaged causing pain or discomfort when moving the injured joints or limbs. There is a need to contain the injury under compression to distribute the load more evenly over the muscles and away from the injury. Thus there is a need for a product that allows the muscles and joints to move, while compressively supporting the injury. For prevention of injury, the compression on the muscles allows enhanced muscle alignment, improving efficiency and efficacy of movement.

Recovery from injury and injury prevention is important for both elite athletes, whose career depends on recovering quickly, and amateur competitors who are trying to make their mark in a sport, or social sports players, whose lifestyle can be affected by injury. Thus, products that speed the recovery of injuries or are able to reduce injuries, also reduce the disruption injuries cause. This can result in the earlier return of a player to the sport, or a reduction in the time taken for an injured person to recover. An industry has been built around sports injuries, with sports medicine being currently recognised as a major branch of medicine. Thus products that can aid recovery or prevent injury are in demand.

It is well known that strapping an injury can assist recovery, by reducing the likelihood of further injury due to the applied compression. Thus compression bandages and tapes are commonly applied to injured joints and limbs. Further compression garments are known to be used by sports players to prevent injury. However, these garments do not provide the targeted protection of strapping.

Compression stockings have been found to increase circulation and reduce limb swelling, which assists in the recovery of injuries and reduction of blood clots. However, these stockings are not focused on the injured area, but merely provide overall compression to the legs.

### DISCLOSURE OF INVENTION

The present invention is directed to a compression garment or method of manufacture thereof which may partially ameliorate or overcome the abovementioned problems or at least provide the consumer with a useful commercial choice.

According to a first broad form of the invention, there is provided a compression garment comprising:
a compression fabric formed into a number of panels; and
a number of seams joining adjacent panels such that the shape of the compression garment is formed by the number of panels attached together at the number of seams;
wherein the shape of the each panel is formed to define of muscle group according to the surface anatomy of the limb or trunk such that the garment compresses the said muscle group and follows the contour of the surface anatomy of the musculature as defined at the seams between adjacent panels when the garment is assembled and worn.

According to a second broad form of the invention, there is provided a method of manufacturing a compression garment comprising the steps of:
laying out a compression fabric;
applying one or more patterns to the compression fabric that defines the surface anatomy of musculature of muscle groups;
cutting the compression fabric into panels according to said patterns; and
sewing the panels together such that a number of seams follow the surface anatomy of the musculature.

Preferably, the seams are flat locked.

Further preferably, the panels define a number of major muscles anatomy groups.

Further preferably, lower body garments include a panel for the groin muscle group.

Further preferably, lower body garments include a panel for the quadriceps muscle group.

Further preferably, longer lower body garments include a panel for the hamstring muscle group.

Further preferably, long lower body garments include a panel for the calf muscle group.

Further preferably, upper body garments include a panel for the trapezius and pectoralis muscle group.

Further preferably, upper body garments include a panel for the abdominal muscle group.

Further preferably, upper body garments include a panel for the oblique and latissimus dorsi muscle groups.

Further preferably, sleeved upper body garments include a panel for the deltoid muscle groups.

Further preferably, longer sleeved upper body garments include a panel for the deltoid and lateral triceps muscle groups.

Preferably, the fabric comprises polyester and elastomeric.

Further preferably polyester is in the range of 60 to 85%, while elastomeric is in the range of 40 to 15%.

Further preferably the ration of polyester to elastomeric is 73.1% to 26.9%.

Further preferably, the fabric is graduatedly compressed.

Further preferably, the fabric contains a multidirectional muscle fit hyper compressive weave.

Further preferably, the fabric has wick properties to disseminate moisture.

In one embodiment, the garment is a one piece body suit.

In another embodiment the garment is a pair of pants.

In another embodiment the garment is a pair of shorts.

In another embodiment the garment is a top.

In another embodiment the garment is a vest.

### BRIEF DESCRIPTION OF DRAWINGS

The invention will now be described in reference to the following drawings in which:

FIG. 1 shows isometric views of two whole body garment according to a first embodiment of the invention;

FIG. 2 shows a method of manufacturing a compression garment according to a second embodiment of the invention;

FIG. 3 shows a front, back and side view of a lower body garment according to a third embodiment of the invention;

FIG. 4 shows a front, back and side view of a lower body garment according to a fourth embodiment of the invention;

FIG. 5 shows a front, back and side view of a lower body garment according to a fifth embodiment of the invention;

FIG. 6 shows a front, back and side view of a lower body garment according to a sixth embodiment of the invention;

FIG. 7 shows a front and back view of an upper body garment according to a seventh embodiment of the invention;

FIG. 8 shows a front and back view of an upper body garment according to an eighth embodiment of the invention;

FIG. 9 shows a front and back view of an upper body garment according to a ninth embodiment of the invention;

FIG. 10 shows a front, back and side view of an upper body garment according to a tenth embodiment of the invention;

FIG. 11 shows a front, back and side view of an sock garment according to a eleventh embodiment of the invention; and

FIG. 12 shows a front, back and side view of a whole body garment according to a twelfth embodiment of the invention.

### BEST MODE FOR CARRYING OUT THE INVENTION

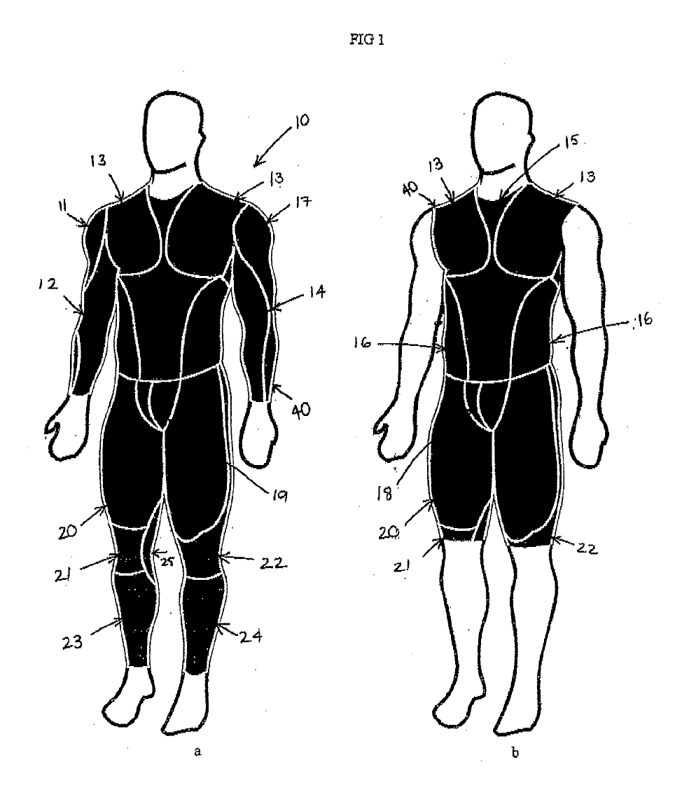

As can be seen in FIG. 1, the invention is a garment 10 for reducing or preventing soft tissue injury comprising a compression fabric formed into a number of panels with a number of seams joining adjacent panels, the shape of the each panel is formed to define of muscle group according to the surface anatomy of the limb or trunk such that the garment compresses the said muscle group and follows the contour of the surface anatomy of the musculature as defined at the seams between adjacent panels when the garment is assembled and worn.

In FIG. 1 a, the garment is a full body garment 10 covering all limbs of the body with openings at the neck, wrists and ankles. Nineteen panels are shown from the isometric view, with the rear view and therefore the rear panels hidden. These panels follow the outline of particular muscle groups to provide targeted compressive support, isolating each muscle group from the other. Thus, when a muscle is flexed or extended, the fabric remains around that isolated group and moves with the flexion or extension. Thus the isolated support allows targeted compression to be applied to each muscle group even when the wearer of the garment is in motion/active.

In this representation, the panel shapes and seams correspond to nineteen of the muscle groups in the isometric view. Each fabric panel is represented in black and the seams are shown in white. On each arm, an outer panel 11 defines the deltoids, triceps and flexors, while an inner panel 12 defines the rest of the arm, including biceps and extensors. The chest area includes two panels 13, 14 shown to define the left and right pectoralis muscles and extending to the dorsal trapezium. The central chest panel 15 defines the abdominis group from the neck to the waist, with lateral panels 16, 17 on each side defining the obliques. The lower regions start at the waist, with two panels 18, 19 defining the left and right groin regions. The top of the legs include a panel 20 defining the quadriceps between the waist and the upper knee. The patella regions are also defined by panels 21, 22 enclosing all of the musculature of each knee. Each shin region has a panel 23, 24 defining the peroneus muscle group. Two panels 25, 26 only one of which is visible define the dorsal region of each knee.

The advantage of this design is that the muscle groups are isolated by the panels and seams so that compression is applied to individual muscle groups, allowing the fabric to operate as a sheath surrounding each muscle group. This allows the garment to move with the muscle groups in flexion and extension. Thus the garment encloses each region like a muscle sheath, strengthening and providing support to the targeted muscle groups, rather than acting like skin which covers the whole body surface without targeted support.

The panels are assembled in a way to provide targeted compression to the relevant muscle groups for each panel. This targeted compression allows each muscle group to be independently supported thus ensuring any injury is kept under compression even during movement of the musculature. For use in injury prevention, the alignment of the muscles the translational movements of the muscles are reduced, while the linear movements are promoted, thereby improving muscle efficacy. This leads to increased efficiency in movement and reduces the likelihood of injury due to muscle misalignment. Also the use of individual panels reduces the push-pull effect of skin type compression garments, which move as a whole rather than responding to a specific muscle group.

The targeted compression allows for both injury recovery and injury prevention. Injuries are understood to include, muscle, tendon and ligament tears, strain and physiological stresses, minor discomfort, major injuries and fatigue.

To understand soft tissue injuries it is important to consider the physiology of exercise. When exercising, the muscles require extra oxygen and energy. The body goes through three cycles of glucose metabolism to produce this energy, namely anaerobic glycolysis for short energy bursts (eg 100 m dash, 25 m swim), glycosis for medium energy bursts—producing the bi-product of lactic acid (eg 400 m run, 50 m swim) and aerobic glycolysis for long distance events (eg marathon, 1500 m swim). The first of these cycles provides about 15 seconds of energy, and the second provides about 30 to 40 seconds of energy before the third cycle begins. The body temperature rises as the amount of oxygen used increases and the oxygen available for cooling drops, leaving a person hot at the end of the exercise session.

If an activity falls within the glycosis range, the person will have “heavy” muscles from the build up of lactic acid. This dissipates as the lactic acid is broken back down into pyruvic acid with time. Injuries occur during this cycle due to pushing the muscle beyond endurance when lactic acid is present. With short sprints, the muscle is used to maximum physical strength in a short amount of time, thereby over straining the amplitude of the muscle contractions. If an injury occurs here it is usually due to too much strength being required from the muscle over a short period of time causing tetanus of the muscles. Alternatively the muscle strain during the distance metabolism is due to fatigue from low levels of strength being exerted over long periods of time. In this case, the muscle fatigues due to the time spent exercising rather than the strength exerted. Injuries usually occur in the first or second cycle due to over-exertion or fatigue during the lactic acid build up. Delayed Muscle Soreness occurs when the muscles are being used in an unusual way resulting in strains for instance after doing a new sport, or after an extended break from a sport.

By improving the blood flow and general circulation, additional oxygen and nutrients are delivered to the muscles, while waste products such as lactic acid are removed from the muscles. Thus the threshold for lactic acid build up is reduced, and oxygen uptake is increased, thereby lowering the body temperature and reducing the likelihood of muscle fatigue. Additionally, increased oxygen levels assist with delayed muscle strain, allowing the muscles to heal faster. Targeted compression improves the flow of oxygen and thereby improves the circulation. This results in decreased lactic acid, thereby reducing the risk of injury from over straining.

The gastrocnemius and the soleus form the calf muscle allowing plantar flexion of the foot. When these muscles become fatigued, for example during sports, ruptures commonly occur where the Achilles tendon merges with the belly of the calf muscle. Thus by cutting the fabric to form a continuous panel around the calf, the fabric creates a gradient compression which enhances the circulation by increased oxygen flow and therefore assists in quicker lactic acid removal, reduced overheating, thereby reducing delayed muscle soreness. In effect this allows an athlete to get more out of their body due to the increases in efficiency provided by the extra oxygen.

By grouping muscles together in one panel, muscle and ligament strain is reduced as muscles are kept in line and at optimal positions with applied compression. Additionally, muscle vibration is reduced by the sheath further optimizing muscle alignment.

Similarly the hamstrings are made up of the semitendinosus, semimembranousus and biceps femoris which create flexion of the knee and extension of the thigh. As these muscles operate as a group they have been contained within the one panel for optimal alignment to prevent injury or assist in recovery from injury.

The quadriceps muscles are made up of rectus femoris, vastus medialis, vastus intermedius and vastus lateralis which allow extension of the leg and flexion of the thigh. These have also been treated as one group with a single panel supporting them for optimal alignment.

Further the groin muscles of pectineus adductor, adductor brevis, adductor magnus and adductor longus have also been treated as a special muscle group with panels defining the left and right groin regions. This is especially important for males who often sustain groin injuries, but less important for females. Thus in the case of garments designed for females there may be no panels for the groin region.

The composition of the fabric, is within the range of 60 to 85% polyester or nylon or similar material combined with 40 to 15% elastomeric material to provide the adequate level of compression against the muscles. The preferred ratio is around 75% polyester or nylon or similar material and around 25% elastomeric material. This compression improves the circulation and thereby assists with recovery and reduces the likelihood of injury occurring. Other suitable compression fabrics can be used to provide the same result as will be understood by a person skilled in the art. For example, carbon based fabrics may be substituted for the same result.

The fabric creates a graduated compression across the musculature by virtue of the multidirectional tension. The warp and weft recovery load allows compression to be applied in a number of directions. Due to the multidirectional muscle fit hyper compression, the muscles are compressed in a number of directions, thereby improving the ability of the fabric to absorb the impact (dampen) muscle vibration.

Additionally, the fabric can wick moisture quickly, allowing it to be evaporated from the body and thereby increase the thermal properties of the garment. This assists in the reduction of chafing, as moisture build up is reduced. The fabric also has a SPF 50+ rating to reduce the exposure of a competitor's skin to UV radiation in sunlight.

FIG. 1 b shows the invention in the form of a short full body suit. Similarly to FIG. 1 a with the chest panel shapes and seams corresponding to the muscle groups defining the left and right pectoral muscles 13, 14 up to the dorsal trapezium. Other chest panels 15 define the abdominis group, and each lateral oblique group 16, 17. From the waist downwards, a left and right groin region is defined by two supporting panels 18, and 19. The quadriceps are divided from the hamstring muscle group by panel 20 and a seam along the side of each leg.

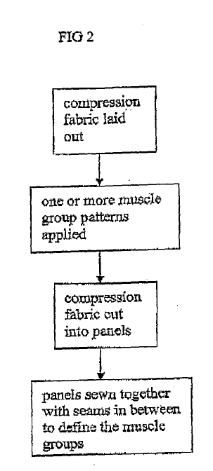

FIG. 2 illustrates a method of manufacturing a compression garment for injury reduction comprising the steps of:

laying out a compression fabric;

applying patterns to the compression fabric that coincides with the surface anatomy of musculature of a number of muscle groups;

cutting the compression fabric into panels according to said patterns; and

sewing the panels together such that a number of seams define the surface anatomy of the musculature.

The patterns applied to the compression fabric defines the number of muscle groups required. The figures show various formations of panels and seams defining the muscle groups required.

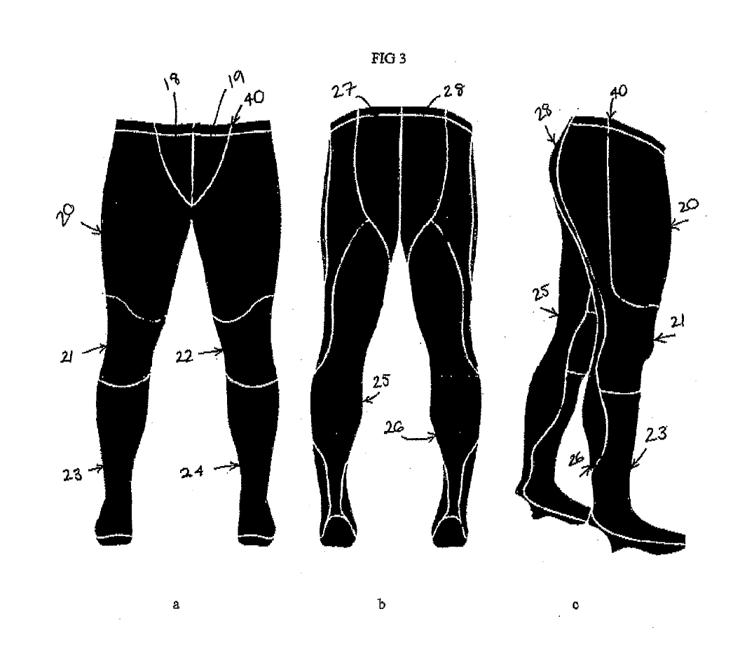

FIG. 3 shows a front view (a) rear view (b) and side view (c) of full length lower body garment designed for improving circulation. As can be seen in the front view, the panels and seams are formed to define the left 19 and right 18 groin regions. The top of the legs include a panel 20 defining the quadriceps between the waist and the upper knee. The patella regions are also defined by panels 21, 22 enclosing all of the musculature of each knee. Each shin region has a panel 23, 24 defining the peroneus muscle group. Two panels 25, 26 define the dorsal region of each knee. Two further panels 27, 28 enclose each leg ending with a seam between the gluteal muscles. This configuration is designed for increased blood circulation and ideal for travel flying, driving, running, cold feet, muscular support, workers who spend long hours on their feet (eg nurses, doctors, police, retail workers, bar staff etc).

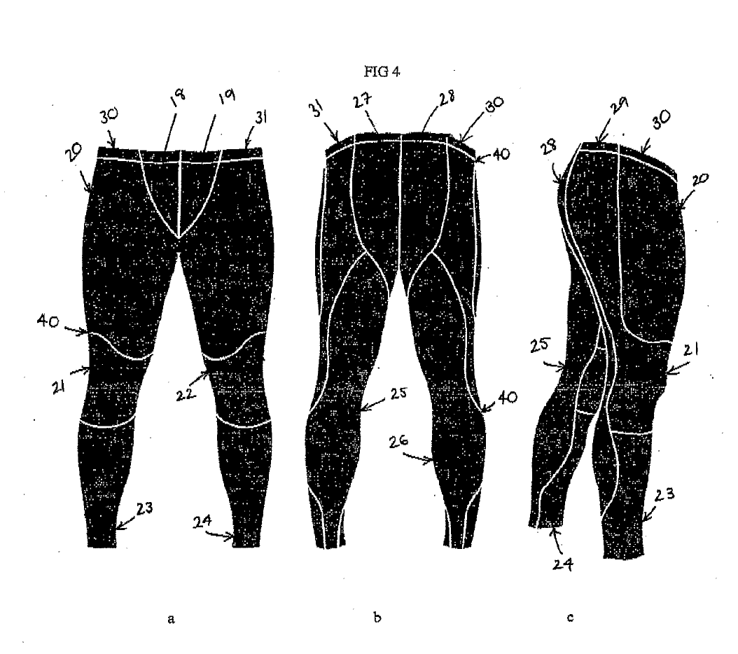

FIG. 4 shows a front view (a) rear view (b) and side view (c) of full length lower body garment. This configuration contains twelve panels representing twelve distinct muscle groups. As can be seen in this view, from the waist downwards, two panels 18, 19 with associated seams are formed to define the left and right groin region. Another two panels 20 define the left and right quadriceps on the anterior upper surface of each leg. As best shown in the side view, another two panels 21, 22 define the gluteus medius for each leg extending into the patella region. Another two panels 27, 28 define the gluteus maximus for each leg, as best shown in the rear view. The hamstring, soleus and also gastrecnominus, for each leg are defined together in another two panels 25, 26 as shown in the rear view. The final two panels 23, 24 define the shin muscle groups for each leg as shown in the front view. Although a number of panels 29, 30, 31, 32 are shown on the waist band, this can be provided as one or more panels.

This garment is useful for targeted compression of muscles either in the lower, upper or entire leg. This configuration is ideal for contact sports, outdoor sports, field sport, gymnastics, track and field, snow skiing, water skiing, sports aquatics, rowing, skating, racing, riding, cycling, athletic, martial arts, team sports, motor sports, and rock climbing.

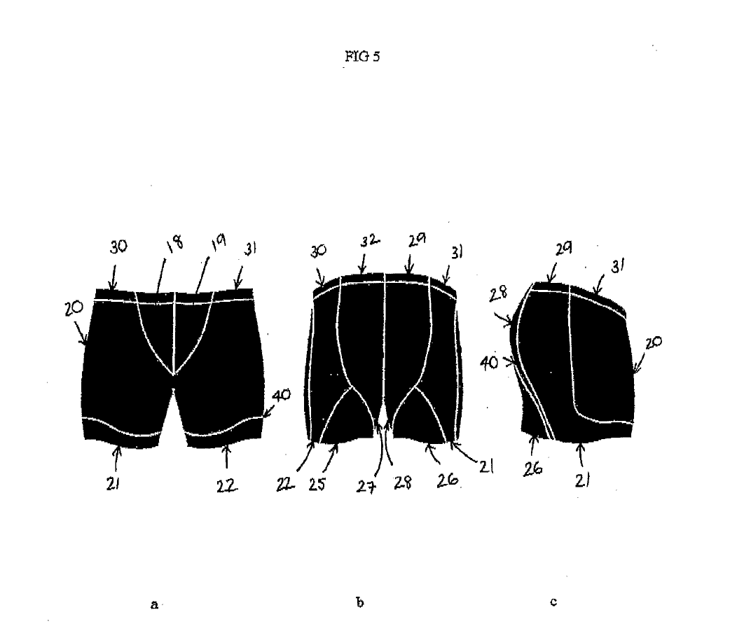

FIG. 5 shows a front view (a) rear view (b) and side view (c) of short lower body garment. As can be seen in this view, the panels and seams are formed to define ten panels. The first two panels 18, 19 define the left and right groin regions, as shown in the front view. The front view also shows that upper quadriceps are defined in a panel 20 for each leg. The side view shows each leg has a panel 21, 22 defining the gluteus medius and extending into the anterior in the middle of the quadriceps. The rear view shows two panels 27, 28 are used to define the gluteus maximus. As also shown in the rear view, another two panels 25, 26 define the top of the hamstring muscle groups for each leg.

This configuration is ideal for sports where a shorter garment is require, or only the groin or upper leg needs support. Also this form of garment allows for targeted muscle compression on upper leg regions between the hip and mid thigh. Examples of sports where this garment would be useful are contact sports, outdoor sports, field sport, gymnastics, track and field, snow skiing, water skiing, sports aquatics, rowing, skating, racing, riding, cycling, athletics, martial arts, team sports, and rock climbing.

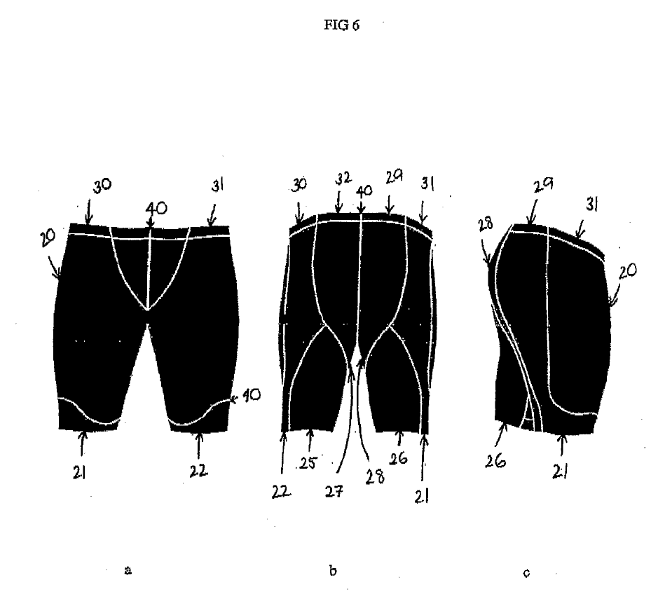

FIG. 6 shows a front view (a) rear view (b) and side view (c) of another short lower body garment. As can be seen in this figure, the panels and seams are formed to define ten panels too. The first two panels 18, 19 define the left and right groin regions, as shown in the front view. The front view also shows two panels 20 defining the left and right quadriceps extending from the waist anteriorly to the knee. The side view shows there are panels 21, 22 for each leg defining the gluteus medius and extending into the anterior enclosing the upper patella region. The rear view shows two panels 27, 28 are used to define the gluteus maximus and gracilis. Another two panels 25, 26 define the top of the hamstring muscles groups for each leg up to the dorsal region of the knee as seen in the rear view.

This configuration is ideal for contact sports, outdoor sports, field sport, gymnastics, track and field, snow skiing, water skiing, sports aquatics, rowing, skating, racing, riding, cycling, athletics, martial arts, team sports, and rock climbing. This form of garment allows for targeted muscle compression on upper leg regions between the hip and knee.

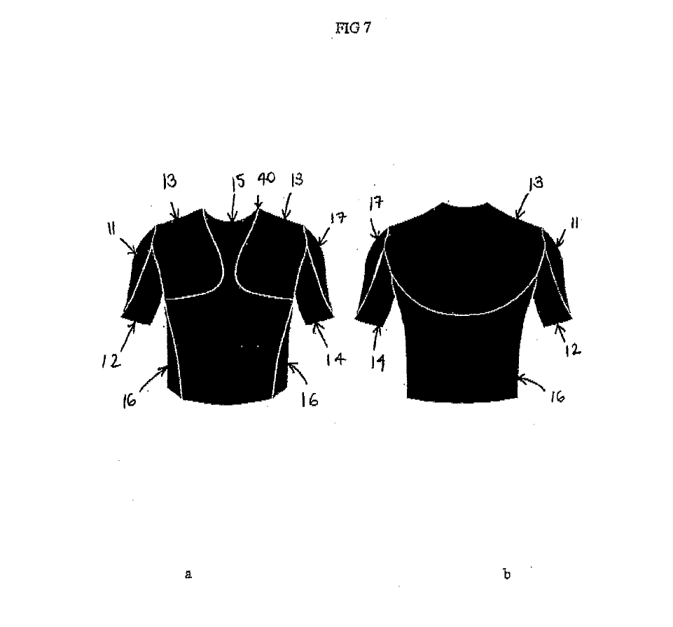

FIG. 7 shows a front view (a) and rear view (b) of a short sleeve upper body garment. As can be seen in this view, there are seven panels with associated seams. The first panel 13 is formed to define the left and right pectoral muscles, both deltoids and lateral triceps and the anterior and dorsal trapezium, with the teres majors. Two panels 11, 17 define the biceps of the left and right arms. Two more panels 12,14 are used for the remaining arm muscles defining the medial triceps and biceps up to mid-arm. The front view shows the anterior abdominal group, defined in another panel 15 extending from the neck to the waist. The final panel 16 defines the lateral obliques and latissimus dorsi (lumbar back) muscle groups.

This form of garment allows for targeted muscle compression on upper body regions between the waist, the neck and shoulder. This configuration is ideal for contact sports, outdoor sports, field sport, gymnastics, track and field, snow skiing, water skiing, sports aquatics, rowing, skating, racing, riding, cycling, athletic, martial arts, team sports, and rock climbing.

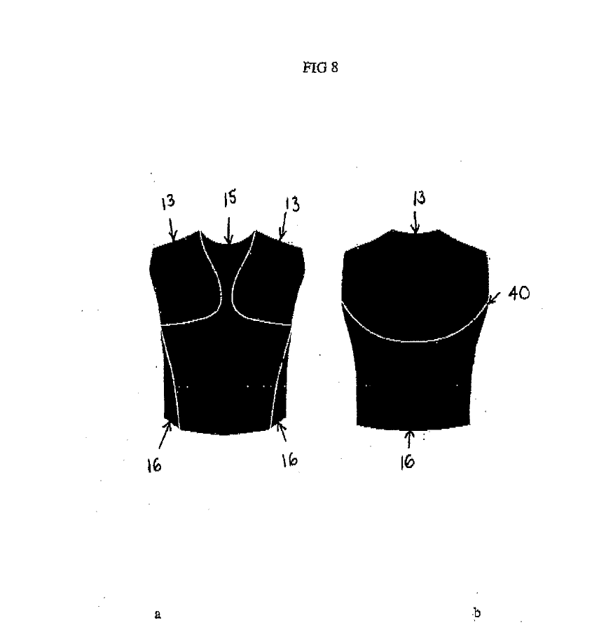

FIG. 8 shows a front view (a) and rear view (b) of another sleeveless upper body garment. As can be seen in this view, three panels and seams are formed to define the garment. The first panel 13 corresponds to the left and right pectoralis and dorsal trapezius with the teres majors. The second panel 15 defines the anterior abdominis group from the neck to the waist. The third panel 16 is defined for the lateral obliques, and latissimus dorsi muscle groups.

This form of garment allows for targeted muscle compression on upper body regions focusing on the chest. This configuration is ideal for contact sports, outdoor sports, field sport, gymnastics, track and field, snow skiing, water skiing, sports aquatics, rowing, skating, racing, riding, cycling, athletic, martial arts, team sports, and rock climbing.

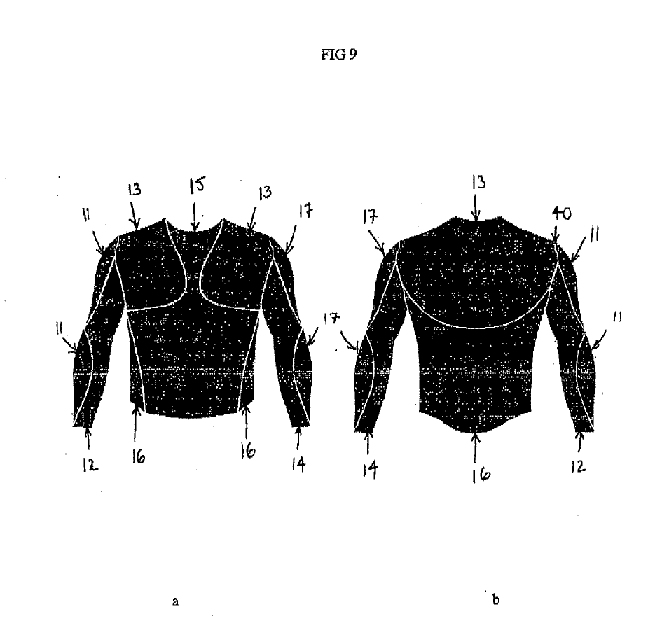

FIG. 9 shows a front view (a) and rear view (b) of a long sleeve upper body garment. In this garment there are seven panels with associated seams. The views show, a first panel 13 defining the left and right pectoralis and anterior and dorsal trapezius with the deltoids and flexors for each arm. Two panels 11, 17 define the biceps of the left and right arms. Two more panels 12,14 are used for the upper arm muscles defining the medial triceps and biceps up to mid-arm. The front view shows the anterior abdominal group, defined in another panel 15 extending from the neck to the waist. The final panel 16 defines the lateral obliques and latissimus dorsi (lumbar back) muscle groups.

This form of garment allows for targeted muscle compression on upper body regions especially on the lower arms. This configuration is ideal for contact sports, outdoor sports, field sport, gymnastics, track and field, snow skiing, water skiing, sports aquatics, rowing, skating, racing, riding, cycling, athletic, martial arts, team sports, and rock climbing.

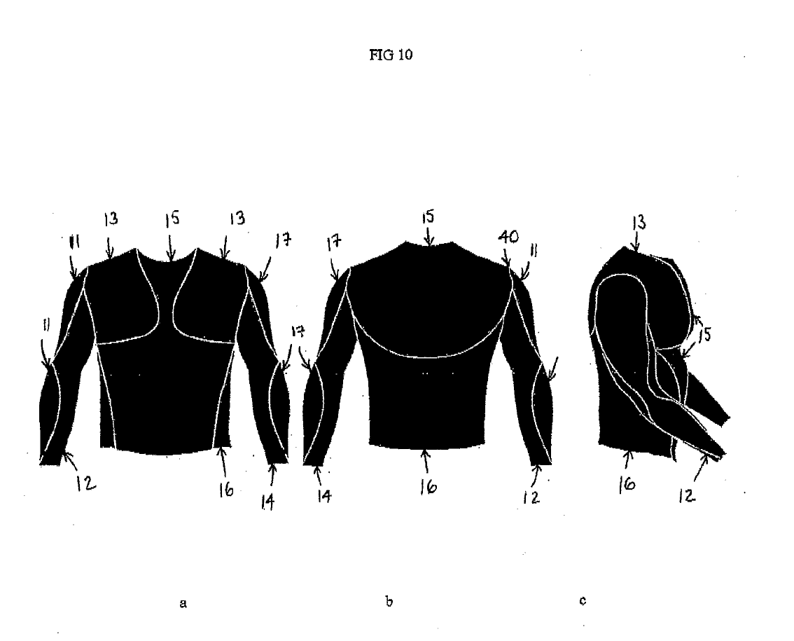

FIG. 10 shows a front view (a) and rear view (b) of the long sleeve upper body garment shown in FIG. 9 with a longer hem to provided additional waist coverage. FIG. 10 (c) shows an isometric view of the panels indicating how panel 11 and 17 correspond to the shape of the arm.

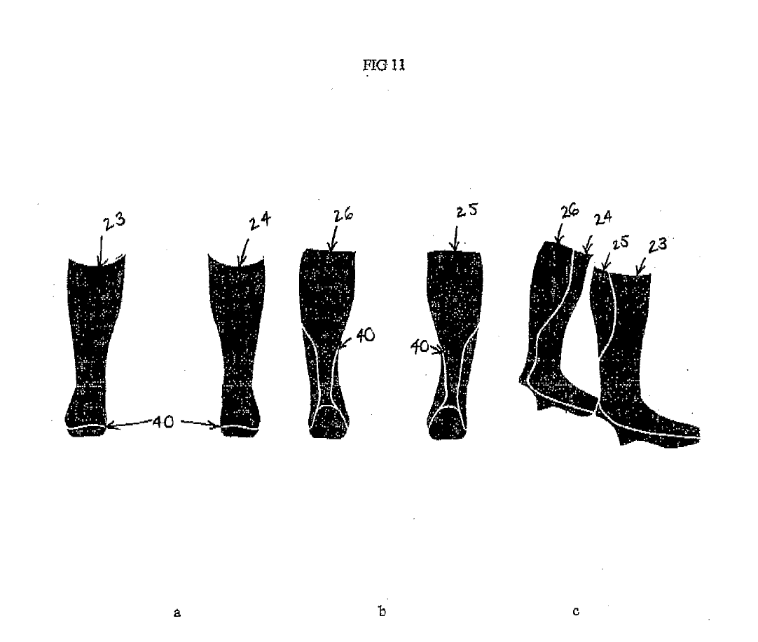

FIG. 11 shows a (a) rear view (b) and side view (c) of a sock garment which applies targeted compression to the lower legs, with a panel 23, 24, 25 and 26 for each of the front and back calf regions of each leg. This garment is used to target the lower legs.

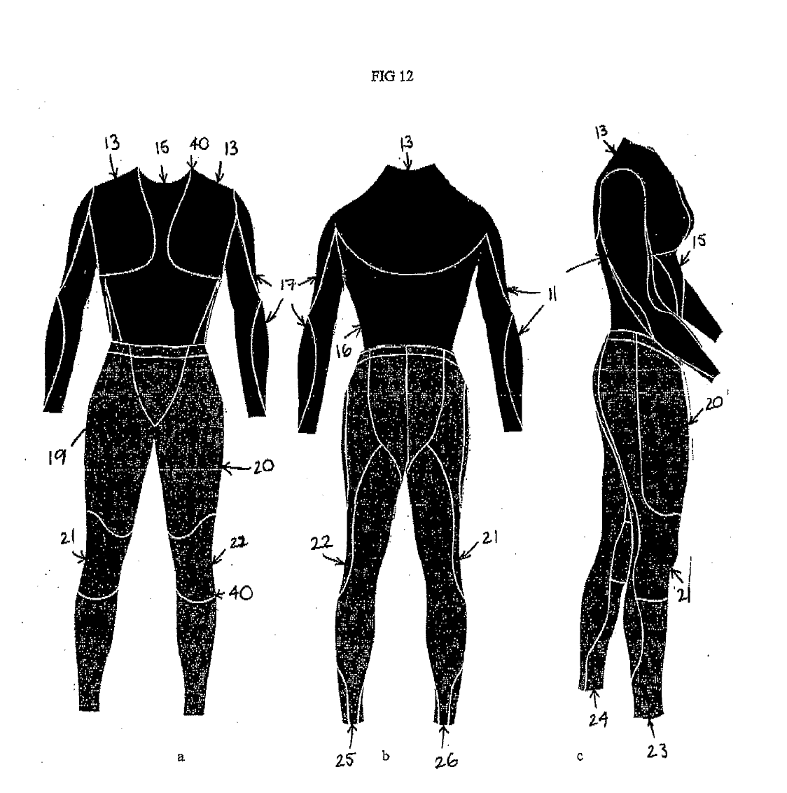

FIG. 12 shows a front view (a) rear view (b) and side view (c) of the long sleeve upper body garment shown in FIG. 1 for a female, with a single groin panel.

By wearing the garment during training, pre-training, post training and during matches, the garment can either prevent or reduce likelihood of injury or assist in a quicker recovery of the player.

It will be evident to a person skilled in the art that numerous modifications and variations of the present invention are possible in light of the above teachings. It is therefore understood the invention may be practiced otherwise than as specifically described herein.

The invention claimed is:

1. A compression garment comprising:
a compression fabric formed into a number of panels, said panels defining muscle groups according to the surface anatomy of a person; and
a number of seams joining adjacent panels;
such that the shape of the compression garment is formed by joining adjacent said number of panels together at said number of seams;
wherein each garment panel provides targeted compressive support to the said muscle groups isolating each muscle group from the others and the seams follow the contour of the surface anatomy of the muscle groups when the garment is assembled and worn.

2. The compression garment of claim 1 wherein each garment is formed to allow compression in dynamic and static conditions.

3. The compression garment of claims 1 or 2 wherein the fabric uses gradient compression to isolate the muscle groups.

4. The compression garment of claims 1 to 3 wherein the seams are flat locked.

5. The compression garment of claims 1 to 4 wherein the panels define a number of major muscles anatomy groups.

6. The compression garment of claims 1 to 5 wherein the fabric comprises polyester and elastomeric.

7. The compression garment of claims 1 to 6 wherein polyester is in the range of 60 to 85%, while elastomeric is in the range of 40 to 15%.

8. The compression garment of claims 1 to 7 wherein the ratio of polyester to elastomeric is 73.1% to 26.9%.

9. The compression garment of claims 1 to 8 wherein the fabric is graduatedly compressed.

10. The compression garment of claims 1 to 9 wherein, the fabric contains a multidirectional muscle fit hyper compressive weave.

11. The compression garment of claims 1 to 10 wherein the fabric has wick properties to disseminate moisture.

12. The compression garment of claims 1 to 11 wherein the garment is a pair of shorts.

13. The compression garment of claims 1 to 10 wherein the garment is a pair of pants.

14. The compression garment of claims 1 to 13 wherein the lower body garments include a panel for the groin muscle group.

15. The compression garment of claims 1 to 14 wherein lower body garments include a panel for the quadriceps muscle group.

16. The compression garment of claims 1 to 15 wherein longer lower body garments include a panel for the hamstring muscle group.

17. The compression garment of claims 1 to 11 wherein long lower body garments include a panel for the calf muscle group.

18. The compression garment of claims 1 to 11 wherein the garment is a top.

19. The compression garment of claims 1 to 11 wherein the garment is a vest.

20. The compression garment of claims 1 to 11 wherein upper body garments include a panel for the trapezius and pectoralis muscle group.

21. The compression garment of claims 1 to 11 wherein upper body garments include side panels for the abdominal muscle group.

22. The compression garment of claims 1 to 11 wherein upper body garments include a panel for the oblique and latissimus dorsi muscle groups.

23. The compression garment of claims 1 to 11 wherein, sleeved upper body garments include a panel for the deltoid muscle groups.

24. The compression garment of claims 1 to 11 wherein longer sleeved upper body garments include a panel for the deltoid and lateral triceps muscle groups.

25. The compression garment of claims 1 to 24 wherein the garment is a one piece body suit.

26. A method of manufacturing a compression garment comprising the steps of:
laying out a compression fabric;
applying one or more patterns to the compression fabric that define the surface anatomy and musculature of muscle groups;
cutting the compression fabric into panels according to said patterns; and
sewing the panels together such that a number of seams follow the surface anatomy of the musculature.
